(19) United States
(12) Patent Application Publication
(10) Pub. No.: US 2007/0015707 A1
(43) Pub. Date: Jan. 18, 2007

(54) Neuroprotective effect of activin in animals exposed to hypoxia/ischemia

(75) Inventors: Peter David Gluckman, Remuera (NZ); Christopher Edward Williams, Grafton (NZ); Dahao Wu, Mt. Roskill (NZ); Paul Edmund Hughes, Blockhouse Bay (NZ); Maggie Lai, Canonmills (GB)

Correspondence Address: FLIESLER MEYER, LLP, FOUR EMBARCADERO CENTER, SUITE 400, SAN FRANCISCO, CA, 94111, US

(73) Assignee: Neuren Pharmaceuticals Limited, Grafton (NZ)

(21) Appl. No.: 11/493,883

(22) Filed: Jul. 26, 2006

Related U.S. Application Data
(63) Continuation of application No. 10/157,542, filed on May 28, 2002.
Application No. 10/157,542 is a continuation of application No. 09/508,897, filed on Mar. 21, 2000, now abandoned.

(30) Foreign Application Priority Data
Sep. 19, 1997 (NZ) 328,796

(51) Int. Cl. A61K 38/17

(52) U.S. Cl. 514/12

(57) ABSTRACT

This invention includes embodiments comprising activin and analogs of activin for neuronal rescue in patients having neurons that are subject to death and/or degradation as a result of neurological diseases. In certain embodiments, an activin receptor agonist can be useful for treating neuralogical diseases such as Alzheimer's Disease, Parkinson's Disease or other diseases involving neural degeneration and loss of neural function.

This invention is directed to a new therapeutic use of activin and its analogs. More particularly, it is directed to the use of activin and its analogs as neuronal rescue agents and/or neuronal phenotype restoratives.

### BACKGROUND

Members of the transforming growth factor (TGF)-β family display multiple roles as hormonal, paracrine and autocrine regulators of cellular function, growth and differentiation. Amongst them, activin is emerging as an important factor in an increasingly diverse range of biological processes including hormone production and secretion, modulation of testicular and ovarian cell function, induction of erythropoiesis, initiation of early embryonic development and more recently as a promoter of wound repair.

As another example of its various properties, activin has been shown to be neuro-prophylactic and to promote survival of neurons subjected to a subsequent toxic event (see, for example, Krieglstein et al; “TGF-β superfamily members promote survival of midbrain dopaminergic neurons and protect them against MPP⁺ toxicity” EMBO Journal Vol. 14 No. 4 pp 736-742 (1995)). However, there has been no suggestion to date that activin has application as a neuronal rescue agent.

As used herein, neuronal “rescue” is distinct from “prophylaxis”. A neuronal “rescue” agent is one which, when administered after an insult prevents neurons from dying which would otherwise be destined to die. In contrast, a neuro-prophylactic agent is one which protects neurons against insult but only where the agent is present at the time of or before the insult.

It is the applicants finding that activin is a neuronal rescue agent as well as being neuro-prophylactic which primarily underlies the present invention.

A number of known compounds are neuro-prophylactic but are not neuronal rescue agents, eg. flunarazine (Gunn and Gluckman, (1991)), GM1 ganglioside (Simon et al., (1993)), NGF-β (Rabizadeh et al., (1994)), bFGF (Mattson et al., (1993)), TNFα (Barger et al., (1995)) and TNF (Barger et al., (1995)). Other compounds are neuronal rescue agents without being neuro-prophylactic, eg. IGF-1 (Guan et al., (1993), Gwag et al., (1995)) and BDNF (Gwag et al., (1995)). Still a further group of compounds are both (for example GPE). The fact that a compound is neuro-prophylactic therefore cannot be predictive of that compound being effective as a neuronal rescue agent.

### SUMMARY OF THE INVENTION

In a first aspect, the present invention provides a method of treating a patient to rescue neurons otherwise destined to die as the result of a prior neuronal insult which comprises administering to said patient activin or an analog thereof after said insult in an amount sufficient to prevent the neurons from dying.

In a further aspect, the present invention provides a method of treating a patient to rescue neurons otherwise destined to die as the result of a prior neuronal insult which comprises increasing the active concentration of activin within said patient after said insult such that the neurons are prevented from dying.

“Neuronal insult” is used herein in its broadest possible sense and includes neuronal insults due to trauma, toxins, asphyxia, hypoxia-ischemia (HI) and disease.

“Activin” as used herein means activin A, activin B or activin AB of mammalian origin and preferably of human, porcine, bovine or murine origin.

“Analog” is used herein to mean a variant of activin through insertion, deletion or substitution of one or more amino acids but which retains at least substantially equivalent biological activity to activin.

The applicants have found that the neuronal rescue role of activin is mediated through the activin type II receptor. Specifically contemplated analogs are therefore those which bind to and activate the activin type II receptor.

In a further aspect, the invention provides a method of treating a patient to rescue neurons otherwise destined to die as the result of prior neuronal insult which comprises activating the activin type II receptors of neuronal cells of a patient who has suffered a prior neuronal insult.

Activation can be through administration of a ligand which binds to, and activates, the receptor.

Preferably, activin type II receptor activation is effected through administration of activin or an analog thereof.

In still a further aspect, the invention provides the use of activin or an analog thereof, or a ligand which binds to and activates activin type II receptors, in the preparation of a medicament for rescuing neurons otherwise destined to die as a result of a prior neuronal insult.

In a further aspect, the invention provides a method of treating a patient to restore the phenotype of neurons degenerating as a result of a prior neuronal insult which comprises administering to said patient activin or an analog thereof after said insult in an amount effective to restore the phenotype of said neurons.

In still a further aspect, the invention provides a method of treating a patient to restore the phenotype of neurons degenerating as a result of a prior neuronal insult which comprises increasing the active concentration of activin within said patient after said insult such that the phenotype of said neurons is restored.

In yet a further aspect, the invention provides a method of treating a patient to restore the phenotype of neurons degenerating as a result of a prior neuronal insult which comprises activating the activin type II receptors of neuronal cells of a patient who has suffered a prior neuronal insult.

In still yet a further aspect, the invention provides the use of activin or an analog thereof, or a ligand which binds to and activates activin type II receptors, in the preparation of a medicament for restoring the phenotype of neurons degenerating as a result of a prior neuronal insult.

### DESCRIPTION OF THE DRAWINGS

While the present invention is broadly as defined above, those persons skilled in the art will appreciate that it is not limited thereto and that it further includes embodiments of which the following description provides examples. In addition, the invention will be better understood through reference to the accompanying drawings in which:

FIG. 1 is a graph showing the rescue of striatal neurons by activin following HI;

FIG. 2 is a graph showing the rescue of striatal cholinergic phenotype neurons by activin after HI;

FIG. 3 shows the results of immunoreactive staining of the activin-type II receptor in neurons of the striatum (FIG. 3A), the substantia nigra (FIG. 3B), thalamus (FIG. 3C) and paraventricular nucleus (FIG. 3D);

FIG. 4 is a graph showing the rescue of calbindin, cholinergic, NADPH-diaphorase and parvalbumin phenotypic neurons by activin following an intrastriatal quinolinic acid lesion (FIGS. 4A to 4F);

FIG. 5 shows the restorative effects of rhActivinA treatment on striatal interneurons immunostained for ChAT (FIGS. 5A to 5D);

FIG. 6 shows the areas of the rat brain analysed for the effect of activin and inhibin on neuronal rescue (FIG. 6A-cortex and hippocampus; FIG. 6B: striatum);

FIG. 7 shows the comparative effects of activin A and inhibin A on the survival of neurons following HI (FIG. 7A: Activin, FIG. 7B: Inhibin); and

FIG. 8 shows the results of immunohistochemical staining of an Alzheimer's brain FIGS. 8A to 8G).

### DESCRIPTION OF THE INVENTION

As broadly defined above, the present invention relates primarily to neuronal rescue. This is the maintenance of neuronal cells which would otherwise be destined to die as a result of a prior neuronal insult. The cells are therefore “rescued” from death and not merely protected prophylactically.

The invention also relates to phenotype restoration. The applicants have found that degenerating neuronal cells which have lost their phenotype as the result of a prior neuronal insult can be phenotypically restored.

The applicants have found that neuronal rescue\phenotype restoration is able to be effected using two approaches. The first approach is through a focus upon activin. The applicants have found that increasing the effective concentration of activin within a patient following neuronal insult rescues neurons and/or restores their phenotype.

Activin itself is critical to this approach. There are three isoforms of activin, designated activin A, activin B and activin AB. Structural analysis shows that activins are disulphide linked dimers of two subunits, which are two distinct 14 kD β subunits (βA and βB) (Ying, 1989, Vale et al., 1990). Activins (28 kD) are homodimers of the two β subunits. The mature βA or βB subunit has 116 or 115 amino acids respectively including 9 cysteines with no glycosylation sites. The two β subunits share about 85% homology within each species, and are also highly homologous across species. The mature βA subunits are completely identical across porcine, bovine, human and murine species and the mature βB subunit only has differences at four-amino acid positions (Esch et al., (1987)).

Recently, the molecular cloning of activin βC, βD, βE subunits has been reported (Fang et al., 1996). Activins made up from or including one or more of these subunits are in no way intended to be excluded.

All of the above forms of activin are contemplated for use in this invention.

The preferred form of activin for use in this invention is activin A. This is available from National Institute of Health, USA.

Most conveniently, the effective concentration of activin will be increased through direct administration using either activin itself or an activin prodrug (a form which is cleaved within the body to release activin). It is however not the applicant's intention to exclude increasing activin concentration through administration of either activin agonists (substances which effect a direct increase in production or activity of activin within the body, eg. FSH, cAMP [activator of protein kinase A activator], 12-O-tetradecanoylphorbol 13-acetate [TPA, a protein kinase A activator], TGF-β, IL-1β and TNF-α) or inhibitors of activin antagonists (substances which bind activin or otherwise prevent or reduce the action of activin within the body). These latter compounds exert an indirect effect on effective activin concentrations through the removal of an inhibitory mechanism, and include substances such as estradiol.

Follistatin is one such substance. It is a single chain glycosylated protein, which was first isolated from porcine and bovine follicular fluid. The amino acid sequence of follistatin is distinct from those of the activin subunits and any other proteins in the TGF-β family. However, across species, follistatin amino acid sequence is highly conserved with over 98% homology.

As a binding protein for activin, follistatin has been observed to have different actions on the biological activities of activin. Follistatin can directly bind to activin to neutralize its function in many systems (Mathews, 1994). However, it has also been suggested to have an ability to enhance activin action through either bringing activin to its receptors or maintaining a high local concentration of activin. Thus follistatin may exert a dual effect in mediating activin activities (Macconell et al., 1996) as both agonist and antagonist.

Inhibin can also be regarded as an activin antagonist. The mechanism by which this is achieved is not completely understood but is likely to include competitive binding to the activin receptor. Therefore, effecting a decrease in the production or action of inhibin is likely to increase the effective concentration of activin.

Another possibility is administration of a replicable vehicle encoding activin to the patient. Such a vehicle (which may be a modified cell line or virus which expresses activin within the patient) could have application in increasing the concentration of activin within the patient for a prolonged period.

It is also contemplated that activin analogs can be employed in this invention. As used herein, “analog” means a protein which is a variant of activin through insertion, deletion or substitution of one or more amino acids but which retains at least substantial functional equivalency.

A protein is a functional equivalent of another protein for a specific function if the equivalent protein is immunologically cross-reactive with, and has at least substantially the same function as, the original protein. The equivalent can be, for example, a fragment of the protein, a fusion of the protein with another protein or carrier, or a fusion of a fragment with additional amino acids. For example, it is possible to substitute amino acids in a sequence with equivalent amino acids using conventional techniques. Groups of amino acids normally held to be equivalent are:
(a) Ala, Ser, Thr, Pro, Gly;
(b) Asn, Asp, Glu, Gln;
(c) His, Arg, Lys;
(d) Met, Leu, Ile, Val; and
(e) Phe, Tyr, Trp.

Functional equivalency of activin analogs can also be readily screened for by reference to the ability of the analog to both bind to and activate the appropriate receptor. In this case, the receptor is an activin type II receptor.

Similar to TGF-β receptors, there are two types of activin receptors, termed activin type I receptor (ActRI) and activin type II receptor (ActRII). ActRII was the first receptor identified for activin and for other members in the TGF-β superfamily (Mathews and Vale, 1991). The mature ActRII is comprised of 494 amino acids which includes a small 116 amino acid extracellular ligand binding domain, a single transmembrane domain and an intracellular serine/threonine kinase domain, which is common in the TGF-β superfamily. Over 90% sequence homology of ActRII has been observed across species, which is consistent to the high similarity of mature activin PA sequences in various species (Mathews, 1994). A distinct but closely related activin receptor ActRIIB and its four isoforms have subsequently been characterized (Mathews, 1994; Mathews et al., 1992; Attisano et al. 1992). ActRII and ActRIIB are approximately 50-60% identical in the ligand-binding domain and 60-70% identical in the kinase domain. ActRII, ActRIIB and its isoforms all bind to activin with high affinity.

Collectively, ActRII, ActRIIB and their isoforms are referred to herein as “activin type II receptors”.

Activin type II receptors are distinct from Activin type I receptors (ActRI). ActRI and its isoforms have been cloned using PCR with oligonucleotides based on the ActRII sequence. These receptors also have highly conserved serine kinases. However, cells expressing ActRI but not ActRII cannot bind to activin alone (Mathews, 1994) and hence the capacity to bind to activin type II receptors is considered critical to this aspect of the invention.

Activin initiates signal transduction across the membrane through both ActRI and ActRII which can form a stable complex with the ligand (Willis et al., 1996; Mathews, 1994). ActRII binds activin and then associates with a type I receptor. This is followed by auto- and trans-phosphorylation between the two receptors and the initiation of intracellular signalling (Smith J, 1995; Willis et al., 1996).

This leads to the applicant's second approach to neuronal rescue and/or phenotypic restoration. This approach focuses upon activin type II receptors as defined above, and upon effecting neuronal rescue\phenotypic restoration through the use of ligands which both bind to and activate these receptors.

It will be appreciated that activin and its analogs are ligands which achieve this. Indeed, the use of activin and activin analogs represents a preferred aspect of the invention. However, it should be appreciated that this approach is not restricted to the use of activin and its analogs but also extends to any ligand which fulis the functional requirement of both binding to and activating (stimulating) the activin type II receptor. Implicit in this will be the ability of the ligand to effect the association with ActRI needed to initiate intracellular signalling.

Such stimulatory ligands can be identified by a screening protocol employing at least the ligand binding domain of an activin type II receptor. This screening method can, for example, utilize the expression of the Act II receptor in Xenopus oocytes using standard recombinant DNA methods and measurement of the Act II receptor-mediated signal transduction evolved by novel stimulatory ligands. Further classical “grind and bind” ligand-binding experiments can also be utilized. Here, whole brain or specific brain regions would be homogenized and the specific-binding of novel compounds to the Act II receptor characterized. This technique allows further characterization of specificity and affinity (potency) of the compound for the Act II receptor complex.

For the intended therapeutic application, the active compound (activin, analog or ligand) will be formulated as a medicament. The details of the formulation will ultimately depend upon the insult to be remedied and the route of administration, but will usually include combination of the active compound with a suitable carrier, vehicle or diluent.

Insults which can be treated in accordance with the invention include any prior neuronal insult. These include trauma, toxins, asphyxia, ischemia and disease, particularly neurodegenerative diseases such as Alzheimer's disease (including both early and late onset forms) Parkinson's disease, Huntington's disease and Lewy Body disease. Also included is peripheral neuropathy.

To be effective as a neural rescue\phenotype restoring agent in treating the above insults, a variety of administration routes can be used. Examples include peripherally in conditions where the blood brain barrier is disrupted (ie. ischemia), intracerebroventricularly (ICV), intraventricular administration involving neurosurgical insertion of a ventricular cannula with an abdominal pump and reservoir and intraparenchymal (ie. at the site of action).

Dosage rates will also be formulation- and insult-dependent. However, by way of example, the recommended dosage rate of activin A formulated for injection would be in the range of 1 ng/100 g to 100 μg/100 g administered centrally.

The invention, in its various aspects, will now be illustrated by the experimental section which follows. It will however be appreciated that the experiments are non-limiting.

### EXPERIMENTAL

### Experiment 1

Materials and Methods

Animal Preparation

The following experimental protocol followed guidelines approved by the University of Auckland Animal Ethics Committee. HI injury was induced in weaned 21 day old Wistar rats using a modified version of the Levine rat preparation as described previously (Sirimanne et al., (1994)). Rats were maintained on a 12 hour cycle of light and dark and given free access to food and water throughout the study. Rats of both sexes weighing between 40-49 g were selected, anaesthetized with halothane, and underwent double ligation of the right carotid artery following exposure through a midventral neck incision. After surgery rats were allowed to recover in a carefully controlled environment of 34° C. with >80% relative humidity for a period of 2 hours were then exposed to 15 minute 8% PO₂ and then lightly anaesthetized again using Saffan (2 mg/kg), ip). Infusions were made into the right lateral cerebral ventricle with the aid of a metal cap fitted over the head of the anaesthetized animal as described by Jirikowski (1992), to allow correct placement of the infusion needle (30 G×25.4 mm). A randomized block design was used to enable treatment studies to be performed in batches of litter mates of 12-16 infant rats.

Treatment and Tissue Preparation

Recombinant human activin A at a dose of 1 μg or vehicle was administered in a single bolus to rats two hours after hypoxia as described above (Activin A, n=23; vehicle, n=23). Activin was given as a 20 μl infusion at rate of 3 μl/min. Only those rats where the solution flowed were accepted. (=all rats). Rats were euthanised 72 hours after hypoxia by sodium pentobarbital overdose and brains were collected for histological processing after in situ fixation by transcardial perfusion with 30 ml saline (0.9%) then neutral buffered formalin (10%). Fixed brains were dehydrated through graded alcohols, defatted in chloroform and embedded in paraffin. Serial 4 mm sections were then cut between at 3.5 mm (for striatum) anterior to the ear bars [Paxinos] and stained with acid-fuschin/thionin, or with an antibody to choline-acetyltransferase (ChAT) which identifies cholinergic neurons (Boehringer Mannheim).

Double-Labeling Immunohistochemistry

Double immunohistochemical labelling to localise ActRII to specific neuronal subtypes was performed on rat brain sections at the level of striatum (cholinergic, parvalbumin, calbindin) or substantia nigra (dopaminergic). Brains were processed as described above. Antiserum generated towards a synthetic fragment of mActRII (482-494) was a generous gift of Professor Wylie Vale (The Salk Institute, San Diego, Calif., USA). Sections were dewaxed, rehydrated and incubated for 30 minutes in 0.6% hydrogen peroxide to quench endogenous peroxidase activity. Incubation with ActRII antibody (1:1000) diluted in 1.5% normal goat serum and phosphate buffered saline (PBS) was carried out overnight in a humidified environment. PBS-washed sections were then incubated with biotinylated secondary antiserum for 1 hour, washed, then incubated with an avidin-biotinylated horseradish peroxidase complex (Vector Labs, Burlingame, Calif., USA) for a further 1 hour. Signal for ActRII was visualised using 0.05%, 3,3′diaminobenzidine tetrahydrochloride (Sigma Chemicals, St Louis, Mo., USA). Sections were then reincubated with either parvalbumin (1:1000), calbindin D28K (1:1000) or ChAT (1:50, identifies cholinergic neurons) or tyrosine hydroxylase (1:1000 identifies dopaminergic neurons) as described above except that immunostaining for the second antibody was visualised using benzidine dihydrochloride (BDHC, Sigma). Non-specific immunostaining was determined using normal rabbit serum, normal mouse serum and incubating sections in the absence of primary antibodies.

The total number of surviving neurons were counted in the CA1/2 region of the hippocampus, 3 selected areas of the cortex and 4 selected areas of the striatum or the total number of ChAT immunopositive neurons in the dorsolateral striatum were counted in the injured half brain of both treatment groups (vehicle and activin A) with a light microscope (Leica, Germany) and were compared.

Results

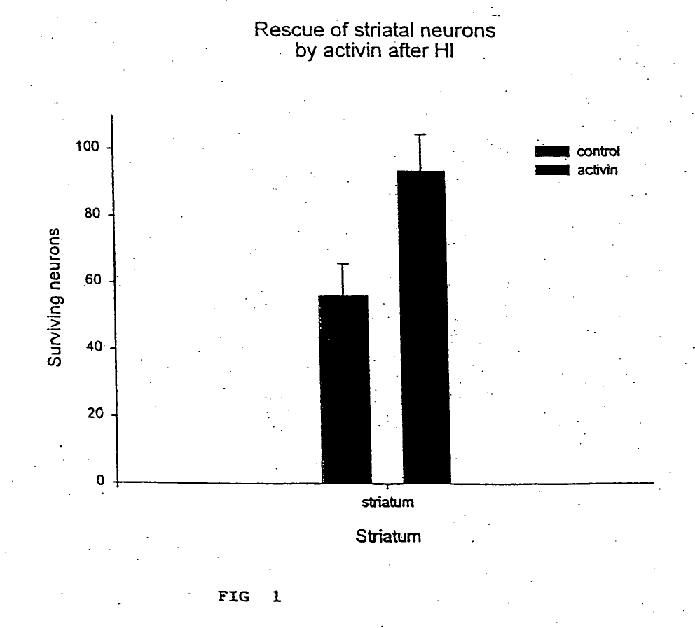

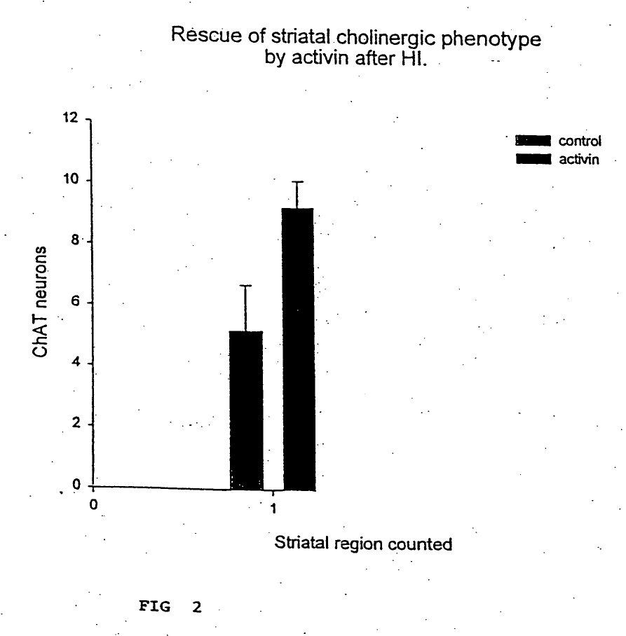

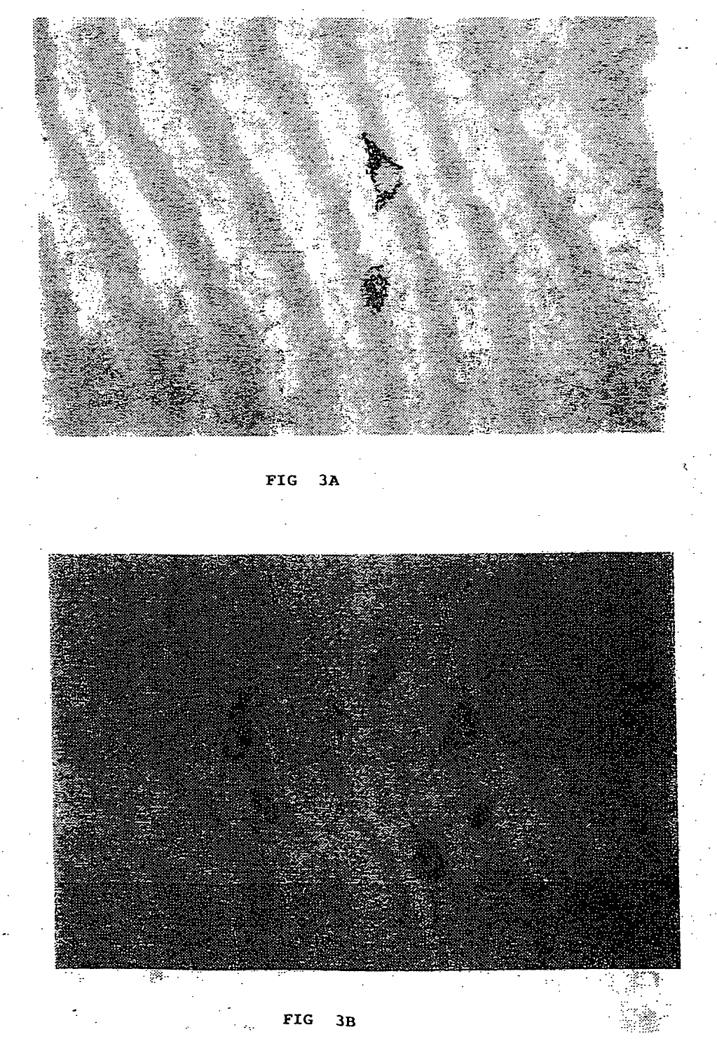

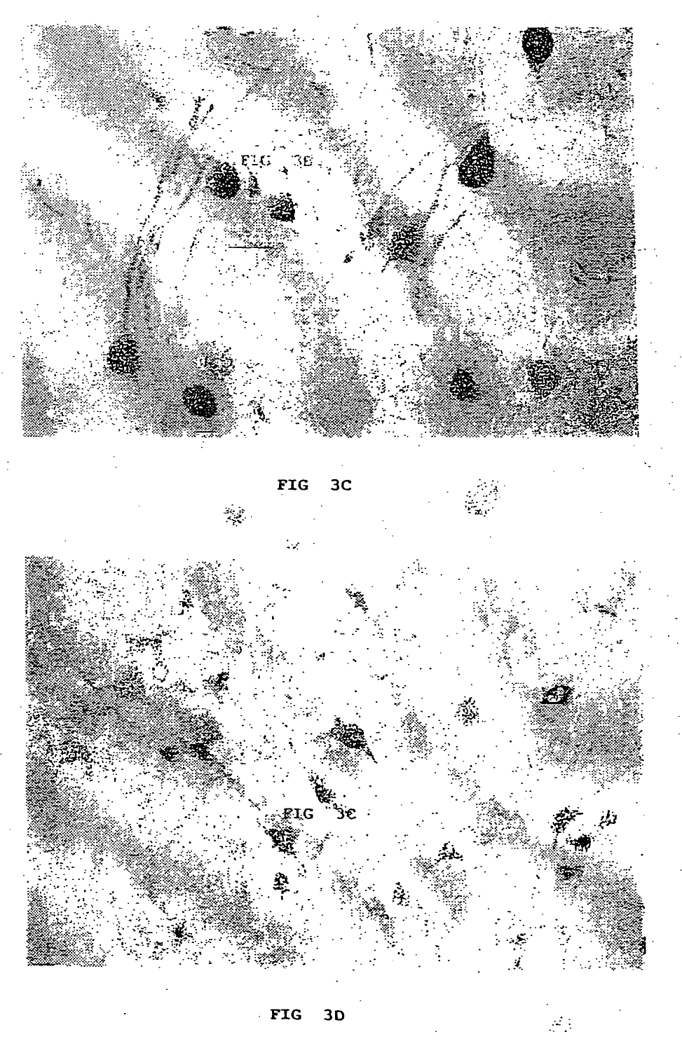

The above experiment provided the following results:
The number of surviving striatal neurons after HI was significantly (p<0.014) increased by activin treatment. This can be seen from FIG. 1.
Activin specifically rescued striatal cholinergic neurons after HI. This can be seen from FIG. 2.
Activin type II receptor immunoreactivity was found in cholinergic, parvalbumin and calbindin neurons of the striatum and in dopaminergic neurons of the substantia nigra. Staining was predominantly in the cell body, axons and processes of neurons but clearly did not show labelling in the nucleus. ActRII immunoreactivity was observed in a number of thalamic nuclei particularly; the lateral and ventral posterior thalamic nucleic, and reticular thalamic nucleus. Staining was also seen in the caudate putamen, layer VI of the cerebral cortex, zona incerta, dorsal lateral geniculate nucleus, and to a lesser intensity in the medial septum and in the hippocampus. This can be seen from FIG. 3.

### CONCLUSIONS

The above results lead to the following conclusions:
1. Activin A is applicable for the treatment of brain injury after HI. Since activin was significantly protective in the striatum and 95% of striatal neurons are GABAergic, activin is likely able to rescue GABAergic neurons in vivo, which has relevance for treating the loss of GABAergic neurons in the striatum seen in Huntington's disease.
2. Activin specifically rescues cholinergic neuronal phenotype after HI and activin type II receptor is colocalized on cholinergic neurons. This indicated an application for activin in treating the hypofunction of central cholinergic neurotransmission seen in the human neurodegenerative condition known as Alzheimer's disease. This further indicated another application for activin in delaying or preventing the loss of cholinergic neurons in the nucleus basalis of Meynert.
3. Activin type II receptor colocalizes with tyrosine hydroxylase (dopaminergic) neurons of the substantia nigra. This indicates an application for activin A in delaying or preventing the loss of dopamine neurons seen in the human neurodegenerative condition known as Parkinson's disease.

### Experiment 2

Methods

The following experiments were carried out in accordance with University of Auckland Animal Ethical Committee Regulations. All efforts were made to keep the number of animals used to a minimum and to minimize animal suffering.

Animal Preparation and Treatment

Two groups (n=4-5) of male Wistar rats weighing 360-400 g (University of Auckland Animal Breeding Unit) were anaesthetized with 75 mg/kg sodium pentobarbital and positioned in a stereotaxic apparatus (Kopf Instruments, USA). Quinolinic acid (QA) lesioning was performed using a modified version of that described by Alexi et al., (1997). Unilateral intrastriatal injections of 100 nmol in 2 μl of QA (Sigma Chemicals) were made over 5 minutes using a Hamilton syringe at the coordinates 0.5 mm anterior to Bregma, 3.0 mm lateral to midline and 5.0 mm ventral to skull from the atlas of Paxinos and Watson (1986). A 22-gauge guide cannula (Plastics One, USA) was permanently fixed into place 0.5 mm dorsal to the ventral coordinate (i.e., at +0.5, +3.0, −4.5). Groups of 4-5 animals received daily 1 μl injections of either rhactivinA (0.73 μg/μl/day, National Hormone and Pituitary Program, CA, USA) or phosphate-buffered saline (PBS, pH 7.4) vehicle beginning at the time of cannulation for 7 days following QA lesioning.

Immunohistochemistry

At 7 days post QA lesioning rats were perfused intracardially with PBS followed by 4% paraformaldehyde in 0.1M phosphate buffer (PB, pH 7.4). Brains were post-fixed overnight at 4° C. in this same solution and cryoprotected serially in 10% and 30% sucrose in PB for 2-4 days at 4° C. Floating 30 μm coronal striatal sections were stained by avidin-biotin-peroxidase immunocytochemistry. GABAergic neurons were stained using an antibody against feline glutamate decarboxylase-67 (GAD₆₇) made in rabbit from Chemicon International (#AB108). Sections were blocked with 10% normal goat serum and 0.25% triton X-100 in PBS for 1 hour at room temperature. Sections were rinsed 3 times in PBS and incubated in primary GAD₆₇ at 1:200 with 2% normal goat serum in PBS at 4° C. for 3 days. Sections were then rinsed and incubated in the secondary antibody, biotinylated anti-rabbit IgG (Amersham RPN480, USA), at 1:1000 with 1% normal goat serum in PBS at 4° C. overnight. Sections were rinsed and incubated in streptavidin biotinylated peroxidase complex (Amersham RPN1050) at 1:100 in PBS at room temperature for 4 hours. Sections were rinsed and developed with diaminobenzidine (Sigma) in deionized water with 0.03% hydrogen peroxide for 5 minutes. Acetylcholine neurons were stained using an antibody against human choline acetyl-transferase (ChAT) made in goat from Chemicon (#AB144P). Sections were stained as for GAD₆₇, except that incubation in the secondary antibody was for 3 hours at room temperature and the solutions were as follows: the blocking solution was 2% rabbit serum in PBS, the primary antibody was diluted 1:100 with 2% rabbit serum in PBS, the secondary antibody was anti-goat at 1:200 in PBS with 2% rabbit serum, the peroxidase complex was AB cocktail (Vector Laboratories, USA) at 9 μl of A plus 9 μl of B per ml of PBS, and the development solution was unchanged. The calcium binding protein containing neurons, calbindin, parvalbumin and calretinin, were stained as for ChAT neurons, except that incubation in the AB peroxidase cocktail was for 1 hour at room temperature and the solutions were as follows: primary antibodies against chicken calbindin (#300) and carp parvalbumin (#235) were made in mouse and human calretinin (#7696) was made in rabbit (Swant Antibodies, Bellinzona, Switzerland), the blocking solution was 10% horse (calbindin and parvalbumin) or goat (calretinin) serum in PBS, the primary antibody was diluted 1:9000 in PBS with 10% respective serum and 0.25% triton X-100, the secondary antibody was anti-mouse or anti-rabbit at 1:250 in PBS, the peroxidase complex and development solutions were unchanged. In order to control for staining intensity, all sections from all the animals for each antibody were processed at the same time. Negative controls consisted of omitting the primary antibody.

Histochemistry

NADPH-diaphorase (NADPHd) cells were visualized by incubating sections in 1 mg/ml NADPH (ICN Biochemicals) and 0.1 mg/ml nitrotetrazolium blue (ICN) dissolved in PB with 0.3% triton X-100 for 2 hours at 37° C.

Quantfication of Cell Counts

Stained cells (ChAT, NADPHd, parvalbumin and calretinin) were counted at 100× magnification in a visual field 950×730 μm. The location of the field was 465 μm below the cannula tip at the lesion center (0.5 mm anterior to Bregma), and extended rostrocaudaly along a parallel to sections at +360 μm rostral to the lesion center and −360 μm caudal to the lesion center. This was done to assess the effects of radial diffusion of solutions from the cannula on the rostrocaudal plane while keeping a fixed distance along the dorsoventral and mediolateral planes. A cell was counted if it had an intact cell body and at least a neurite stump. Cells were counted in a blind coded fashion. Cell counts were converted from the number of cells in the visual field to the number of cells per mm² by dividing by 0.6935 (950×730 μm=0.6935 mm²). Stained cells (GAD₆₇ and calbindin) were counted at 100× magnification in a visual field 475×365 μm. Cell counts were converted from the number of cells in the visual field to the number of cells per mm² by dividing by 0.1734 (475×365 μm=0.1734 mm²). Cell counts are presented as means of cells/mm²±SEM at each of the three levels (rostral, center, caudal) in the striatum for both the contralateral (control) and ipsilateral (lesion and treatment) sides from 4-5 animals per group. To reveal the percentage of cell phenotypic survival, ipsilateral values (cells/mm²) were divided by contralateral values for each animal and means were calculated for each of the two groups. Comparisons between phenotypic cell survival at each coronal level were performed on percentage values to standardize for the differing sizes of the populations examined. Statistical analysis of cell counts were made using a multi-variate analysis of variance (ANOVA) for either repeated (phenotypic marker) or non-repeated (treatment) measures, followed by a post hoc analysis using the Student Newman-Keuls test for statistical differences (Sigma Stat, Jandel Scientific, USA).

Results

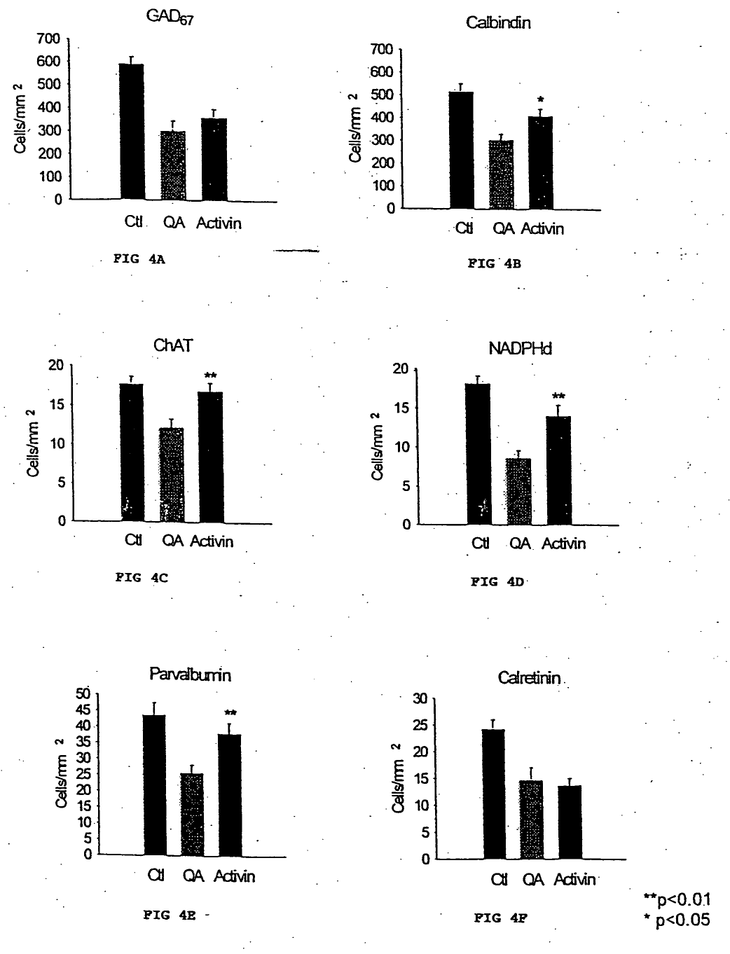

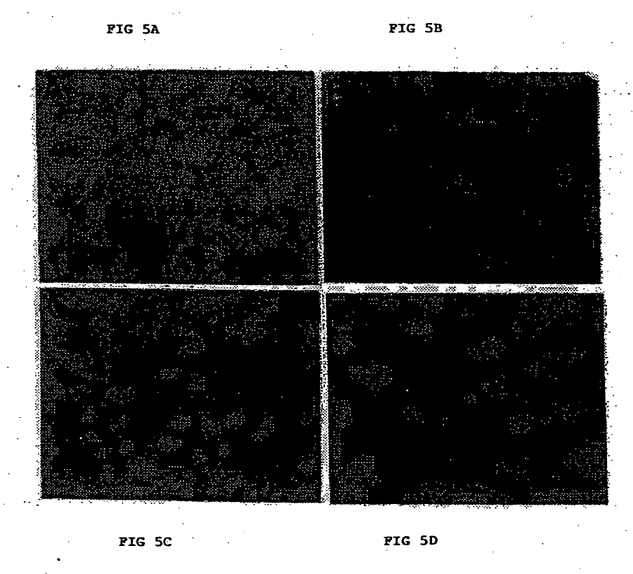

The results are shown in FIGS. 4A-4F and FIGS. 5A-5D.

Unilateral intrastriatal infusion of quinolinic acid produced a partial but significant loss by 7 days in the number of striatal neurons immunoreactive for glutamate decarboxylase (to 51.0±5.8% of unlesioned levels, see FIG. 4A), calbindin (to 58.7±5.1%, see FIG. 4B), choline acetyl-transferase (to 68.6±6.1%, see FIG. 4C), NADPH-diaphorase (to 47.4±5.4%, see FIG. 4D), parvalbumin (to 58.8±4.1%, see FIG. 4E) and calretinin (to 60.6±8.6%, see FIG. 4F) in adult rats that were administered intrastriatal phosphate buffered saline for 7 days following quinolinic acid.

In contrast, in rats that received intrastriatal recombinant human ActivinA once daily for 7 days following quinolinic acid, phenotypic degeneration was significantly attenuated in several populations of striatal neurons. Treatment with ActivinA had the most potent protective effect on the striatal cholinergic interneuron population almost completely preventing the lesion induced decline in choline acetyltransferase expression (to 95.1±5.8% of unlesioned levels, see FIG. 4C). ActivinA also conferred a significant protective effect on parvalbumin (to 87.5±7.7%, see FIG. 4E) and NADPH-diaphorase (to 77.5±7.5%, see FIG. 4D) interneuron populations but failed to prevent the phenotypic degeneration of calretinin neurons (to 56.6±5.5%, see FIG. 4F). Glutamate decarboxylase₆₇ and calbindin-staining nerve cells represent largely overlapping populations and both identify striatal GABAergic projection neurons. ActivinA significantly attenuated the loss in the numbers of neurons staining for calbindin (to 79.7±6.6%, see FIG. 4B) at 7 days following quinolinic acid lesioning.

The results shown in FIGS. 5A-5D illustrate the restorative effects of rhActivinA treatment on striatal stained for CHAT.

Compared with the vehicle treated control (FIG. 5B) the ChAT neurons in both the contralateral striatum control (FIG. 5A) and the QA-lesioned side treated with rhActivinA (FIGS. 5C and 5D) have healthy cell bodies and elongated neuronal processes containing ChAT. FIG. 5D is a high power magnification of FIG. 5C.

### CONCLUSIONS

Exogenous administration of ActivinA rescues both striatal interneurons (labelled with choline acetyltransferase, parvalbumin, NADPH-diaphorase) and striatal projection neurons (labelled by calbindin) from excitotoxic lesioning with QA. It also restores the phenotype of degenerating neurons.

The ability of Activin A to rescue striatal GABAergic projection neurons from degeneration following QA lesioning indicates an application for activin A in delaying or preventing the loss of GABAergic projection neurons which are preferentially lost in the human neurodegenerative disease, Huntington's disease.

These results further indicate that activin A can restore the CHAT neuronal phenotype after a QA lesion in the striatum. This indicates an application for activin A in restoring CHAT neurons in the human neurodegenerative condition Huntington's disease and also Alzheimer's disease.

### Experiment 3

The Effect of Activin A and Inhibin A on Neuronal Survival

Methods

ICV Injection

Moderate HI brain injury (15 minutes hypoxia) was first induced in 21-day-old Wistar rats. Two hours after hypoxia, all rats were lightly anaesthetised with intraperitoneal injection of Saffan. An infusion needle (3 θG×25.4 mm) was placed into the right lateral cerebroventricle of the rat brain with the aid of a metal skull template as described by Jirikowski (Jirikowski, 1992). Each rat was injected either with the drug (activin A or inhibin A) diluted with vehicle solution in a total volume of 20 μl, or 20 ml vehicle solution. The injections into vehicle and treatment groups were performed simultaneously with a micro-infusion pump at a rate of 3 μl/minute. After infusion, the rats were left to recover in an incubator maintained at 34° C. and relative humidity of 85-95%. Once awake, the rats were transferred into their holding cages and fed food and liquid ad libitum.

In the activin A treatment study, 46 rats were divided into two batches, weight and sex matched groups. 23 rats were injected with 1 μg rh activin A diluted with sterilized vehicle solution containing 0.15 M NaCl, 0.05 M Tris and 0.1% BSA, (pH7.4). The other 23 rats were injected with 20 μl vehicle solution.

In the inhibin A treatment study, 36 rats were divided into two groups as above 18 rats were injected with 1 μg inhibin A diluted with sterilized vehicle solution containing 0.9% natural saline, 0.1% BSA, (pH 7.4) and the other 18 rats were injected with 20 W corresponding vehicle solution.

Analysis of Histological Outcome

All rats were euthanised 3 days after ICV injection with an overdose of phenobarbital. The rats were transcardially perfused with 0.9% saline followed by 4% PFA, and the brains removed and embedded in paraffin. Symmetric serial coronal sections (4 mm) were cut and stained with thionin/acid-fusion for live/dead neurons (Sirimanne et al., 1994). The histological outcome of neuronal survival was examined with light microscopy (Leica) in the cortex, hippocampus and striatum in the injured half of the brain according to a reference of rat brain anatomy (Paxinos and Watson, 1982), as these areas suffer most of the neuronal loss in the moderate HI brain injury model (Sirimanne et al., 1994). Only cells with a morphology like live neurons were counted, while dead neurons or cells with morphology like glial were not included. For each of the above three brain areas, one coronal section was used for each brain.

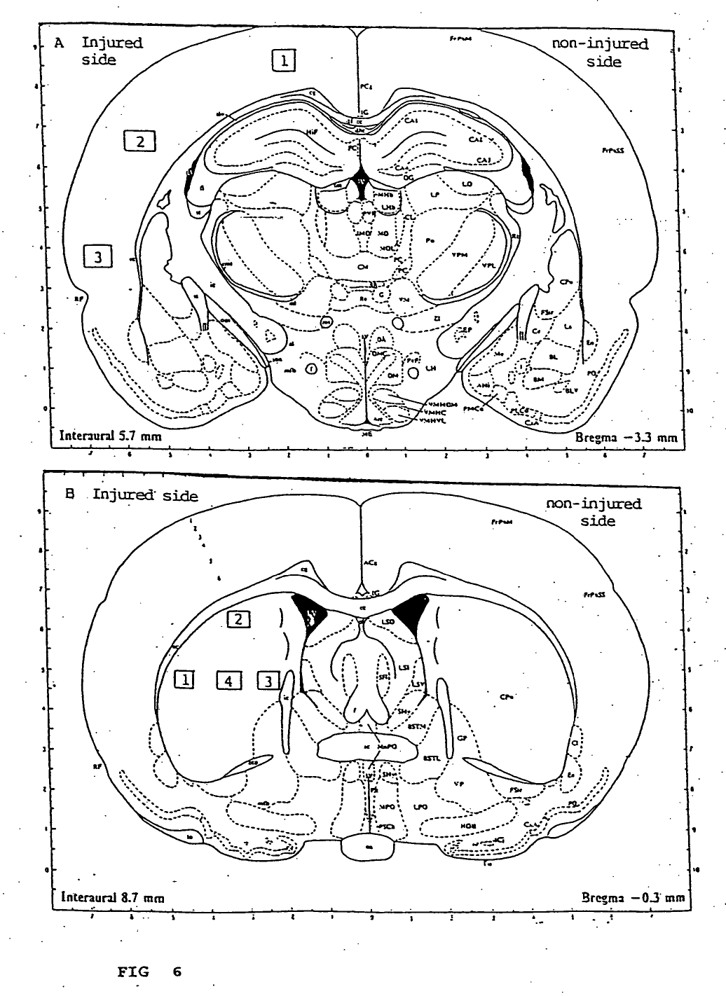

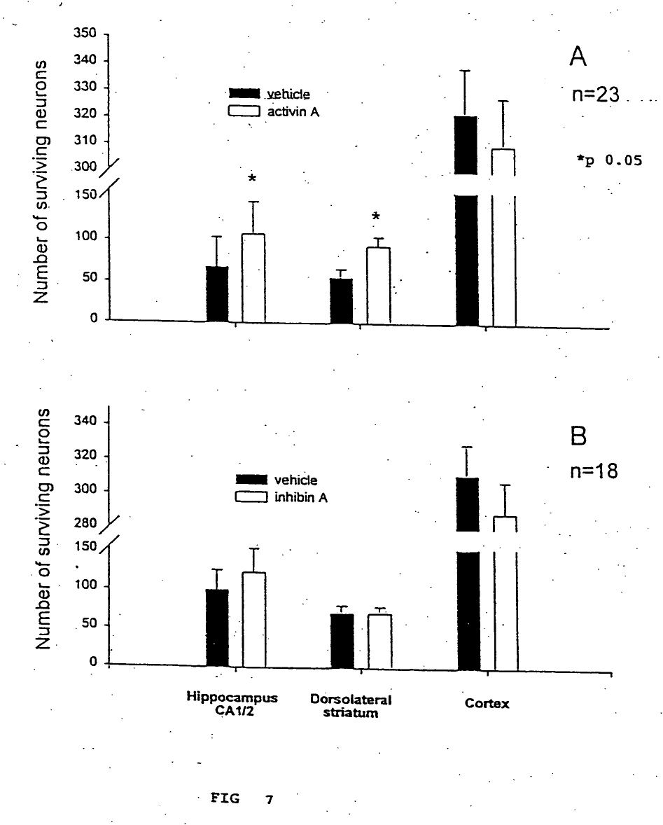

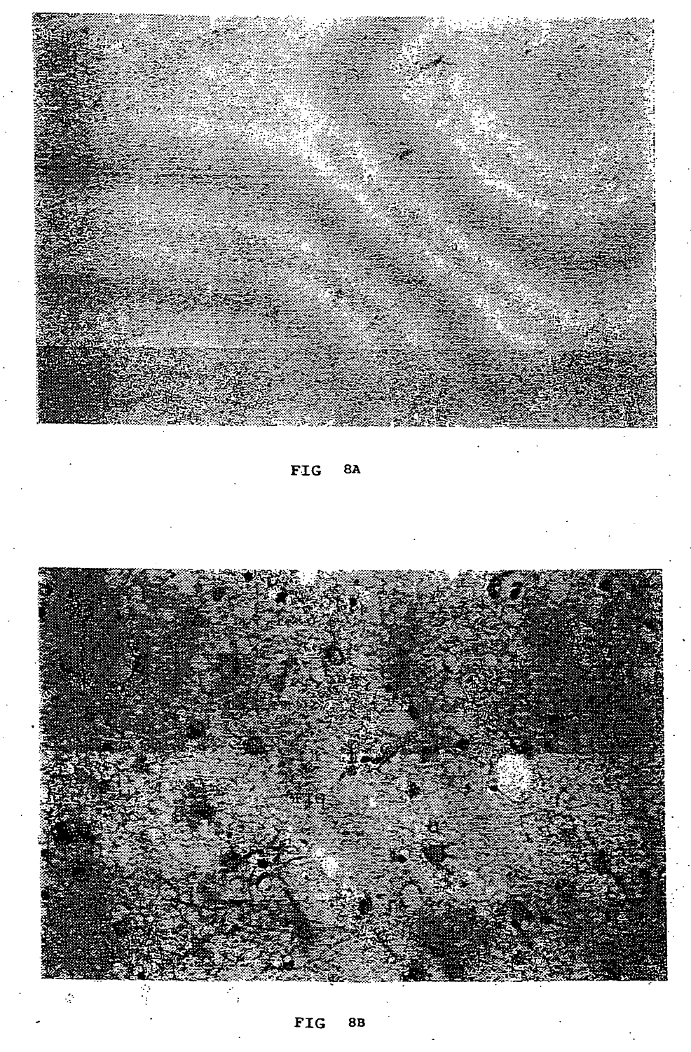

One coronal section between 2.8 mm to 3 mm from Bregma was used for the analysis of cerebral cortex. Live neurons within three rectangular areas each measuring 1000 μm×5000 μm were counted with a microscope grid (FIG. 6A). The areas selected covered layer II to layer V of the parietal cortex, where selective neuronal loss occurred.

One coronal section between 3.3 mm to 3.5 mm posterior to Bregma was used for the hippocampus. All surviving neurons in the hippocampal CA1/2 region of the injured hemisphere were counted (FIG. 6A).

One coronal section between 0.3 to 0.8 mm from Bregma was used for the striatum. Four areas (Areas 1-4) in the upper ⅔ of the injured striatum where most damage is generally found in this model were selected with a microscope grid (FIG. 6B). The size of the areas were: Area 1-3:1000 μm×2000 μm, Area 4:2000 μm×2000 μm.

Results

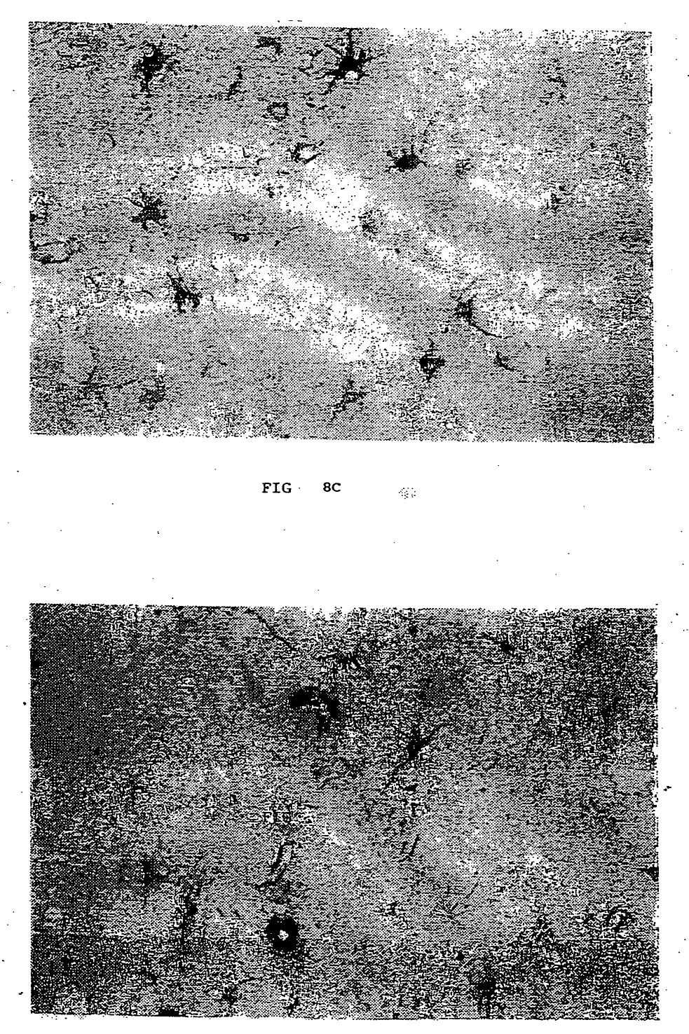

The results are shown in FIG. 7.

In both the treatment and vehicle control group, dead cells identified by the uptake of acid fusion were found in the ligated (right) hemisphere 3 days after hypoxia, particularly in regions such as the CA1/2 region of hippocampus, the upper two thirds of the striatum and layer III-IV of the cerebral cortex. The severity of cell loss ranged from non-selective to massive in these areas. No cell loss was observed in the non-injured hemisphere.

The neural rescue effect of rhActivin A after moderate H1 brain injury was assessed in the hippocampus, striatum and cortex as follows:

Hippocampus

All the live neurons in the hippocampal CA1/2 region in the injured (right) side were counted. In the activin A treated group, the mean number of surviving neurons increased to 107±39 as compared with 67±36 in the vehicle control group (mean±SEM, p<0.05, Mann-Whitney rank sum test).

Striatum

Four representative areas in the dorsal (area 1), lateral (area 2) and central (area 4) region of the upper two thirds of the injured striatum were selected. When the number of live neurons in area 1 and area 2 were added together to represent the dorsolateral striatum, there were more surviving cells in the treatment group (94±11) than in the control group (50±10) (mean±SEM, P<0.05, t-test).

Cortex

The total numbers of live neurons in the selected areas in the parietal cortex in the activin A treated group (310±18) and the vehicle control group (322±17) were not significantly different.

The effect of inhibin A on neuronal survival was also assessed in the hippocampal CA1/2 region, striatum and cerebral cortex as described in the activin A treatment study. The differences of surviving neurons between the treatment and control group in the above three areas were not statistically significant. However, compared with the control group, there was a trend showing that inhibin A decreased the number of surviving neurons in the dorsolateral striatum from 71±10 to 70±9 and in the cortex from 311±18 to 289±19 while increased the number of surviving neurons in the hippocampal CA1/2 region from 100±26 to 123±31 (mean±SEM).

### CONCLUSION

When compared to the results achieved using activin A, it can be clearly seen that inhibin is not effective as a neuronal rescue agent. This is consistent with inhibin binding to but not activating the activin type II receptor and therefore being a functional antagonist of activin. This in turn supports the applicant's findings regarding the critical role activation or stimulation of the activin type II receptor plays in neuronal rescue.

### Experiment 4

Methods

Follistain Expression

The expression of follistatin peptide in the brain of an Alzheimer's sufferer was examined immunohistochemically with a Vectastain kit (Vector labs, USA). Serial formalin fixed and paraffin embedded post mortem human brain tissue (from the medial frontal gyrus) were cut at 4 mm, which were then dewaxed and rehydrated. Non-specific staining which arises from the endogenous peroxidase activity and non-specific binding sites on the brain sections were blocked by 0.3% H₂O₂ and 1% normal goat serum respectively. The sections were incubated in three steps with a polyclonal anti-human follistatin (1:500, a gift from Hiromu Sugino, the Institute for Enzyme Research, The University of Tokushima, Japan) at room temperature ovenight, biotinylated goat-anti-rabbit-IgG (1:100) at 37° C. for 1 hour and horseradish-peroxidase (1:100) at 37° C. for 1 hour. The sections were washed with 0.01M PBS three times after each of the above incubation. The signals were washed with 0.01M PBS three times after each of the above incubations. The signals on the sections were detected with a solution made with DAB tablets (Sigma, USA), which showed a brown colour. After being washed with water, the sections were counterstained with thionin, dehydrated and mounted with DPX.

To identify brain cells which express follistatin, a double immunohistochemistry method was used. The brown immunostaining for follistatin was first obtained on brain sections as described above. As the follistatin positive cells have a morphology similar to glial, the above three step immunostaining procedure with monoclonal anti-GFAP (1:500, Sigma) as the primary antibody was used to identity astrocytes. The signals were detected with BDHC which showed a blue colour.

Cortical senile plaques which contain β-amyloid are characteristic of Alzheimer's disease. To further examine the possible spatial relationship between the follistatin expressing cells and senile plaques, a triple immunohistochemistry method was used. After dewaxing and rehydration, brain sections were treated with concentrated formic acid for 5 minutes, which can enhance the intensity of the immunostaining of senile plaques. The sections were thorough washed with water and blocked for possible non-specific staining. A monoclonal anti-human β-amyloid (1:1000, Dako, Denmark) was used as the primary antibody in the three step immunostaining procedure and Ni-DAB tablets (Sigma) were used as a blue chromagen. The brain sections were then immunostained for follistatin and GFAP as described above.

Activin βA Subunit Expression

Activin βA peptide expression and its spatial relationship to senile plaques was examined with a similar double immunohistochemistry method as described above. Briefly, the brain sections were first immunostained for β-amyloid positive plaques with Ni-DAB as the chromagen. The sections were then incubated with polyclonal anti-activin βA subunit (1:250, a gift from Professor Wylie Vale, The Salk Institute for Biological Studies, USA) as the primary antibody. DAB was used as the brown chromagen for this staining.

Results

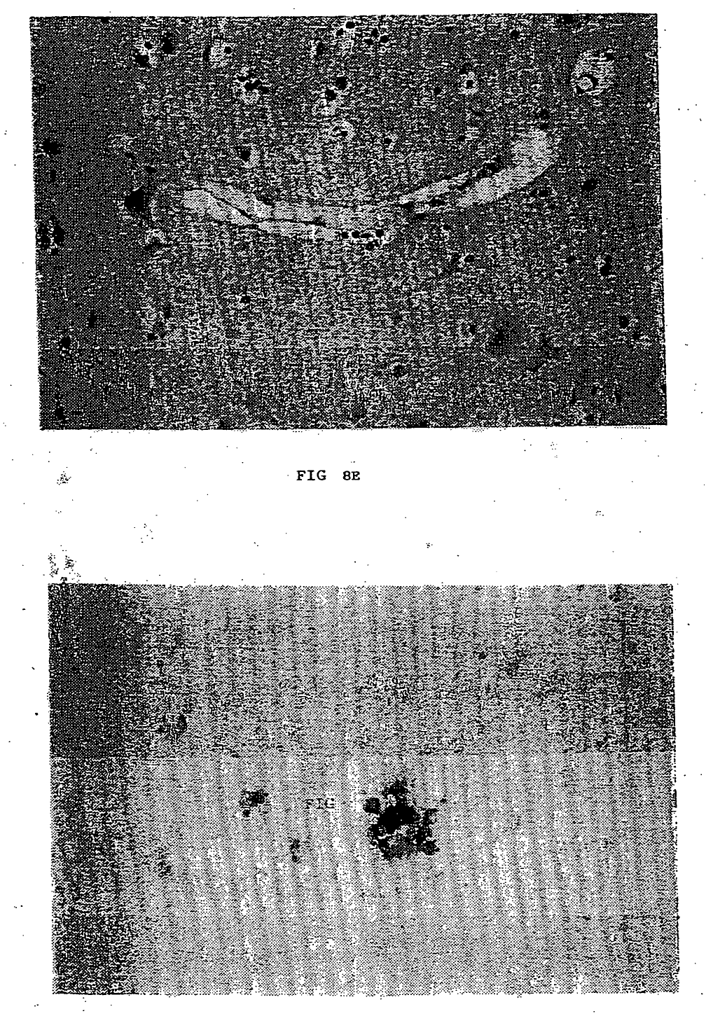

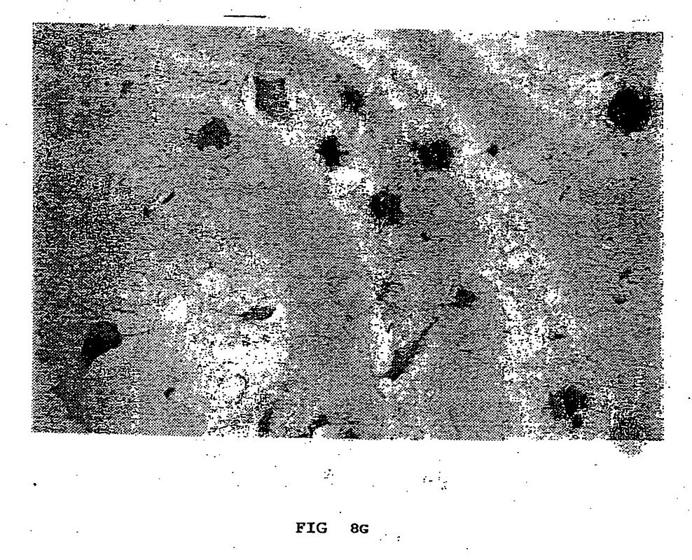

The results are shown in FIGS. 8A-8G.

Compared with an age matched control brain (FIG. 8A), follistatin protein is increased in an Alzheimer's disease patient brain (FIG. 8B). Double immunohistochemistry labelling showed that follistatin expression was mainly colocalised with glial fibrillary acidic protein (GFAP) positive astrocytes (blue staining) and also possibly microglia (FIGS. 8C and 8D). FIG. 8D also shows staining for β-amyloid (red brown staining). PA activin immunoreactivity (brown staining) was also found inside β-amyloid positive plaques (black staining) in the Alzheimer's disease brains (FIG. 8E=control tissue, FIG. 8F=Alzheimer's brain tissue).

FIG. 8G shows activin type II receptor staining in neurons that ‘look’ damaged in Alzheimer's disease brain tissue. The black staining is β-amyloid.

### CONCLUSION

These results indicate that of follistatin upregulation (an activin inhibitor) may have a role in the pathophysiology of Alzheimer's disease, while the upregulation of activin may indicate the induction of an endogenous neuronal rescue mechanism in the Alzheimer's disease brain.

### INDUSTRIAL APPLICATION

The invention therefore provides new approaches to neuronal rescue and neuronal phenotype restoration. These involve firstly increasing the active concentration of activin in a patient following neuronal insult and secondly the activation of the activin type II receptors localized on neuronal cells, again following neuronal insult.

The approaches of the invention have application in the treatment of patients who have suffered neuronal insult particularly as the result of a neurodegenerative disease. Two such diseases of considerable interest are Alzheimer's disease and Parkinson's disease. Patients suffering from these diseases will benefit greatly by a treatment protocol able to rescue damaged and dying neuronal cell populations.

Other applications of the present invention are in the treatment of Huntington's disease and peripheral neuropathy.

Still more generally, the invention has application in the rescue of neurons destined to die following insult in the form of trauma, toxin exposure, asphyxia or hypoxia-ischemia.

In addition to neuronal rescue, the present methods also show the capability of restoring phenotypes in injured, degenerating and diseased neurons, particularly those of the following phenotypes:
ChAT, calbindin, NADPHd, parvalbumin, GABAergic and glutamatergic neurons.

It will be appreciated by those persons skilled in the art that the above description is provided by way of example only and that numerous changes and variations can be made while still being with the scope of the invention as defined by the appended claims.

### REFERENCES

Alexi T., Venero J. L. and Hefti F. (1997) Protective effects of neurotrophin-4/5 and transforming growth factor-α on striatal neuronal phenotypic degeneration after excitotoxic lesioning with quinolinic acid. Neuroscience 78:73-86.
Attisano L., Wrana J. L., Cheifet S., and Massague J. (1992). Novel activin receptors: distinct genes and alternative mRNA splicing generate a repertoire of serine/threonine kinase receptors. Cell, 68:97-108.
Barger S. W., Horster D., Funikawa K., Goodman Y., Kieglestein J., Mattson M. P. (1995). Proceedings of the National Academy of Sciences, USA, 92:9328-9332.
Esch F. S., Shimasaki S., Mercado M., Cooksey K., Ling N., Ying S., Ueno N. X., and Guillemin R. (1987). Structural characterization of follistatin: a novel follicle-stimulating hormone release-inhibiting polypeptide from the gonad. Molecular Endocrinology, 1(11):849-855.
Fang J. M., Yin W. S., Smiley E., Wang S. Q., and Bonadio J. (1996). Molecular cloning of the mouse activin beta(E) subunit gene. Biochemical and Biophysical Research Communications, 228(3):669-674.
Guan J., Williams C. E., Gunning M., Mallard E. C. and Gluckman P. D. (1993). The effects of IGF-1 treatment after hypoxic-ischemic brain injury in adult rats. Journal of Cerebral Blood Flow and Metabolism, 13:609-616.
Gwag B. J., Koh J. Y., Chen M. M., Dugan L. L., Behrens M. M., Lobner D., Choi D. W. (1995). BDNF or IGF-1 potentiates free radical-mediated injury in cortical cell cultures. NeuroReport, 7(1):93-96.
Gunn A. J., Gluckman P. D. (1991). Flunarizine, a calcium channel antagonist, is not neuroprotective when given after hypoxia-ischemia in the infant rat. Developmental Pharmacology and Therapeutics, 17:205-209.
Jirikowski G. F. (1992). A non-surgical technique for accurate intracerebral injections in rat. Journal of Neuroscience Methods, 42:115-118.
Macconell L. A., Barth S., and Roberts V. J. (1996). Distribution of follistatin messenger ribonucleic acid in the rat brain—implications for a role in the regulation of central reproductive functions. Endocrinology, 137:2150-2158.
Matthews L. S., and Vale W. W. (1991) Expression cloning of an activin receptor, a predicted transmembrane serine kinase. Cell, 65:973-982.
Matthews L. S., Vale W. W., and Kinter C. R. (1992). Cloning of a second type of activin receptor and functional characterization in Xenopus embryos. Science, 255:1702-1705.
Mattson M. P., Barger S. W., Cheng B., Lieberburg I., Smith-Swintosky V. L., Rydel R. E. (1993). Beta-amyloid precursor protein metabolites and loss of neuronal Ca²⁺ homeostasis in Alzheimer's disease. Trends in Neurosciences, 16:409-414.
Paxinos G. and Watson C. (1986). The Rat Brain in Stereotaxic Coordinates. 2^{nd} ed. Sydney: Academic Press.
Rabizadeh S., Bittle C. M., Butcher L. L., Bredsen D. E. (1994). Expression of the low affinity nerve growth factor receptor enhances amyloid peptide toxicity. Proceedings of the National Academy of Sciences, USA, 9(22):10703-10706.
Simon R. P., Chen. J. Graham S. H. (1993). GM1 ganglioside treatment of focal ischemia—a dose-response and microdialysis study. Journal of Pharmacology and Therapeutics, 265(1):24-29.
Sirimanne E. S., Guan J., Williams C. E., and Gluckman P. D. (1994). Two models for determining the mechanisms of damage and repair after hypoxic-ischemic injury in the developing rat brain. Journal of Neuroscience Methods, 55:7-14.
Vale W., Hsuch A. J. W., River C. and Yu A. (1990). The inhibin/activin family of hormones and growth factors. In Peptide Growth Factors and their Receptors, Handbook of Experimental Pharmacology. M Sporn and A B Roberts, eds. (Berlin: Springer-Verlag), pp 211-248.
Ying S. Y. (1989). Inhibins, activins and follistatins. Journal of Steroid Biochemistry, 33:705-713.

1. A method of treating a patient to rescue neurons otherwise destined to die as the result of a prior neuronal insult which comprises administering to said patient activin or an analog thereof after said insult in an amount sufficient to prevent the neurons from dying.

2-35. (canceled)

36. A method of treating a patient to protect neurons otherwise destined to die as the result of a hypoxic/ischemic neuronal insult, comprising administering to said patient a pharmaceutically effective amount of activin.

37. The method of claim 36, wherein the activin is selected from activin A, activin B and activin AB.

38. The method of claim 36 wherein the activin administered is activin A.

39. The method of claim 36, wherein said activin is administered before the onset of said hypoxic/ischemic insult.

40. The method of claim 36, wherein said activin is administered after said hypoxic/ischemic insult.

41. The method of claim 36, wherein said hypoxic/ischemic insult is associated with stroke.

42. The method of claim 36, wherein said hypoxic/ischemic insult is associated with coronary artery bypass graft surgery.

43. The method of claim 36, wherein said hypoxia/ischemic insult is associated with decreased blood flow through the carotid artery.

44. The method of claim 36, wherein said hypoxia/ischemic insult is associated with reduced partial pressure of oxygen in the arterial blood of said animal.

45. The method of claim 36, wherein said activin is administered to the central nervous system.

46. The method of claim 36, wherein said activin is administered into a cerebral ventricle.

47. The method of claim 36, wherein said protected neurons are striatal neurons.

48. The method of claim 36, wherein said protected neurons are cholinergic neurons.

49. The method of claim 36, wherein said protected neurons are dopaminergic neurons.

50. The method of claim 36, wherein said protected neurons are in the substantia nigra.

51. The method of claim 36, wherein said protected neurons are gaba-ergic neurons.

52. The method of claim 36, wherein said pharmaceutically effective amount of activin is in the range of about 1 ng/100 g body weight to about 100 μg/100 g body weight of said animal.

53. The method of claim 36, wherein said activin acts via activin type II receptors of neuronal cells.

54. A method of treating a patient to protect neurons otherwise destined to die as the result of a toxic neuronal insult, comprising administering to said patient a pharmaceutically effective amount of activin.
